(19) United States
(12) Patent Application Publication
(10) Pub. No.: US 2016/0122790 A1
(43) Pub. Date: May 5, 2016

(54) PROCESS OF SCALE PRODUCTION AND PURIFICATION OF BACTERIAL CELLULOSE OBTAINED BY GLUCOSE POLYMERIZATION FROM SUGARS OF RENEWABLE SOURCES VIA BIOTECHNOLOGY THROUGH THE PROPAGATION OF GLUCONOACETOBACTER HANSENII LMSPE IN REACTORS AND OBTAINMENT OF PURIFIED CELLULOSE FOR APPLICATION IN HEALTH, PHARMACOTECHNICAL AND COSMETIC DERMATOLOGY AREAS

(71) Applicant: POLISA BIOPOLIMEROS PARA SAUDE LTDA - EPP, Recife (BR)

(72) Inventors: Francisco De Assis Dutra MELO, Recife (BR); Lara Moura AGUIAR, Recife (BR)

(73) Assignee: POLISA BIOPOLIMEROS PARA SAUDE LTDA - EPP, Recife (BR)

(21) Appl. No.: 14/928,075

(22) Filed: Oct. 30, 2015

(30) Foreign Application Priority Data
Oct. 30, 2014 (BR) 10 2014 027203 8

(51) Int. Cl. C12P 19/04

(52) U.S. Cl. CPC C12P 19/04

(57) ABSTRACT

A process of scale production and purification of bacterial cellulose obtained by glucose polymerization from sugars of renewable sources via biotechnology through the propagation of Gluconoacetobacter hansenii LMPSE in reactors and obtainment of the purified cellulose for application in health, pharmacotechnical and cosmetic dermatology areas, which provides the steps of a) preparing the must; b) sterilizing the must; c) inoculating the must; d) propagating; e) brightening) and f) obtaining purified bacterial cellulose product. The process of scale production of bacterial cellulose is obtained in a sealed sterilization and propagation unit, biodigestors with the flotation surface area four times the height.

### BACKGROUND OF THE INVENTION

This patent application focuses on the production and purification of bacterial cellulose that is used to develop products for application in the fields of activity involving: health, pharmacotechnical and cosmetic dermatology.

This specification refers to a patent application which proposes a process to produce and purify, in scale, bacterial cellulose obtained through glucose polymerization having as base sugars of renewable sources through the use of biotechnology, by propagating a microorganism (GLUCONOACETOBACTER HANSENII LMSPE), wherein such process allows the resulting product thereof to have particular use in health, pharmacotechnical and cosmetic dermatology areas.

Processes developed for production and purification of bacterial celluloses aimed to health (medicine), pharmacotechnical and cosmetic dermatology areas are known from the state of the art.

In general, processes for production of bacterial celluloses should meet, regarding the characteristics of the obtained product, rigid criteria regarding their state of purity, microcrystallinity and with respect to production scale.

Processes for production of bacterial celluloses available in this are still in the research phase and have not achieved adequate process of scale production and purification.

The process for production and purification of bacterial cellulose to meet, in scale, the need of nanocellulose to the medicine, pharmacotechnical and cosmetic dermatology areas is a technological innovation in the production line of technical-economic impact.

The process for production of bacterial cellulose that does not achieve scale and high purity level only serves the production of films for wound dressings used as a mechanical barrier and does not meet the requirements for producing different products for specific applications in medicine, pharmacotechnical and cosmetic dermatology areas.

Films produced from non-bacterial, oxidized cellulose, despite its high crystallinity, are used in the biological area as protective barrier to injury and as a drug support, because they do not reach the percentage of microcrystallinity obtained with the bacterial celluloses or nanocellulose.

These are biocompatible and non-toxic products, meeting very effectively the demand for dressings and as a mechanical barrier in epithelial lesions, particularly for external applications. Celluloses composed of microfibrils, if implanted in the organism, are not biotransformed and they do not promote remodeling, differing from nanocelluloses, which are readily biotransformed by occurring remodeling in the deployment site.

The most efficient current production via of nanocellulose is the biotechnological via by propagating bacteria producing microfibrillar cellulose and nanocellulose.

The current processes for the production of bacterial nanocellulose are intended to laboratory production and they are still not in scale of demand. However, it is proven and recognized the importance of pure nanocellulose for application in biological area.

The scale production of bacterial cellulose, or nanocellulose at low cost, represents an innovation of technological and economic impact.

In view of the current state of the art, it is proposed this patent invention application, which aims to achieve a process of scale production and purification of micro- and nanofibrillar fractions of bacterial cellulose obtained from sugar renewable sources derived from sugar cane and also of other renewable sources such as dairy products and coconut water by biotechnological means by Gluconoacetobacter hansenii LMSPE isolated from regional biodiversity.

The process of scale production of bacterial cellulose is obtained in sterilization and propagation sealed unit, biodigestors with the flotation surface area of four times the height (FIG. 2).

The maximum sugar conversion rate in polymer mass, bacterial cellulose, crude wet obtained by this scale production process is similar to that obtained with the Zoogloea sp, which reaches under optimum conditions a maximum sugar conversion rate in crude polymer mass of 76.8%. [2].

The bacterial cellulose is an exopolysaccharide produced by different bacteria and the scale production is associated with the synthesis yield of different bacterial species and physical condition of propagation.

The microorganism Gluconoacetobacter hansenii LMSPE isolated from regional biodiversity used in the production process of bacterial cellulose is characterized as a strain of high nanocellulose productivity.

The production of collected crude polymer mass is directly proportional to the surface of the must in the biodigestors. Therefore, its production meets the physical conditions of propagation and is directly proportional to the biodigestor area.

The option of using biodigestors with the lowest depth and larger surface has a positive impact on production, resulting in greater scale in real time and lower production proportional cost.

The use of a sterilization and propagation sealed unit (FIG. 1) represents greater security, quality control, greater production yield at a lower cost with economic impact.

The pure bacterial cellulose is obtained in scale from the crude polymer mass by means of a sequence of treatment which provides the steps of: washing of polymer mass in flowing water bath until said mass reach a light brown coloring; reduction of residual sugars by brightening with the treatment of polymeric mass in a dilute solution of sodium hypochlorite until a translucent mass of pearlescent white coloring is obtained; washings in distilled water bath until complete elimination of traces of sodium hypochlorite. The process allows the scale production of stable bacterial cellulose with high purity, identified by the absence of free glucose in the polymeric mass and by obtainment 97.8% of pure glucose obtained by thermal acid hydrolysis of the dehydrated clarified cellulosic mass analyzed by HPLC.

The process of production, treatment and purification of bacterial cellulose of this process results in a cellulose with high concentration of micro- and nanofibrils with a percentage of crystallinity of 98.8% that meets the specificities and requirements of purity and microcrystallinity suitable for the production of different products for application in medicine, pharmacotechnical and cosmetic dermatology areas in scale demand.

From the purified polymer mass, different products can be obtained for specific applications in medicine, pharmacotechnical and cosmetic dermatology areas. The production and purification of bacterial cellulose that enables its use in different products with application potential in medicine, pharmacotechnical and cosmetic dermatology in scale demand is a scientific and economic technical impact innovation.

This description refers to process of scale production and purification of bacterial cellulose obtained from sugars renewable sources, particularly derived from sugar cane, in addition to other renewable sources too, as dairy products and coconut water, by biotechnological way by Gluconoacetobacter hansenii LMSPE isolated from regional biodiversity for multiple applications, particularly in medicine, pharmacotechnical and cosmetic dermatology areas.

The process of bacterial celulose production obtained in scale by propagation of microorganism isolated from regional biodiversity Gluconoacetobacter hansenii LMSPE in sterilization and propagation sealed unit, biodigestors with a flotation surface area of four times the height (FIG. 1) results in a scale production reaching the Maximum sugar conversion rate in crude polymer mass of 76.8%.

The scale production of bacterial cellulose from sugars renewable sources with high conversion rate is an innovation with an impact on industrial and economic context. For application in medicine, pharmacotechnical and cosmetic dermatology areas, the purification of polymer mass or crude bacterial cellulose is required.

The process of purification consists of washing in flowing water, brightening and reduction of residual sugars by the action of sodium hypochlorite and washing with sterile deionized distilled water.

The final product, purified bacterial cellulose, consists of a translucent mass with pearlescent White coloring with high concentration of nanocellulose, which can be used as a matrix for the production of multiple products for application in medicine, pharmacotechnical and cosmetic dermatology areas. The purified and dehydrated bacterial cellulose is composed of 97.8% of polymerized glucose determined by HPLC by hot acid hydrolysis.

The homopolymer composition of the purified bacterial cellulose consists of 97.8% of glucose from purified bacterial cellulose represents a material com high purity level. The scale production of bacterial cellulose from sugars of renewable sources via biotechnology by Gluconoacetobacter hansenii LMSPE isolated from regional biodiversity in a sterilization and propagation sealed unit is a controlled production process which represents an innovation of technical and scientific impact and market interest by multiplicity and magnitude of its applications in biological area, particularly in medicine, pharmacotechnical and cosmetic dermatology areas.

### SUMMARY OF THE INVENTION

The process consists of scale production of bacterial cellulose from sugars of renewable sources via biotechnology by Gluconoacetobacter hansenii LMSPE isolated from regional biodiversity in sterilization and propagation sealed unit. Washing of crude polymer mass in flowing water until obtainment a product of gelatin aspect of light brown coloring.

After washing, the polymer mass passes through a brightening process which consists of reduction of residual sugars through the action of sodium hypochlorite at a concentration, which may range from 0.3% to 3.0% in a volume from 5 to 10 times the mass volume of the bacterial cellulose ensuring quality to the end product.

The process is concluded in a period from 6 to 36 hours when the product reaches a translucent and pearlescent coloring.

The purification is completed with distilled water washing to remove traces of sodium hypochlorite, which are readily identified when the potential of ionic hydrogen of the washing water reaches pH value of 6.2. After the process of purification, the purified bacterial cellulose may be used to the production of different products for application in medicine, pharmacotechnical and cosmetic dermatology areas such as hydrogels, films, sponges and composites.

### BRIEF DESCRIPTION OF THE DRAWINGS

The process object of this patent invention application will be fully understood through the detailed description, which will be made based on the figures listed below, in which:

FIG. 1 illustrates a flowchart of the process itself.

FIG. 2 illustrates, in a schematic representation, the sterilization and propagation sealed unit used in the process hereby treated.

### DETAILED DESCRIPTION OF THE INVENTION

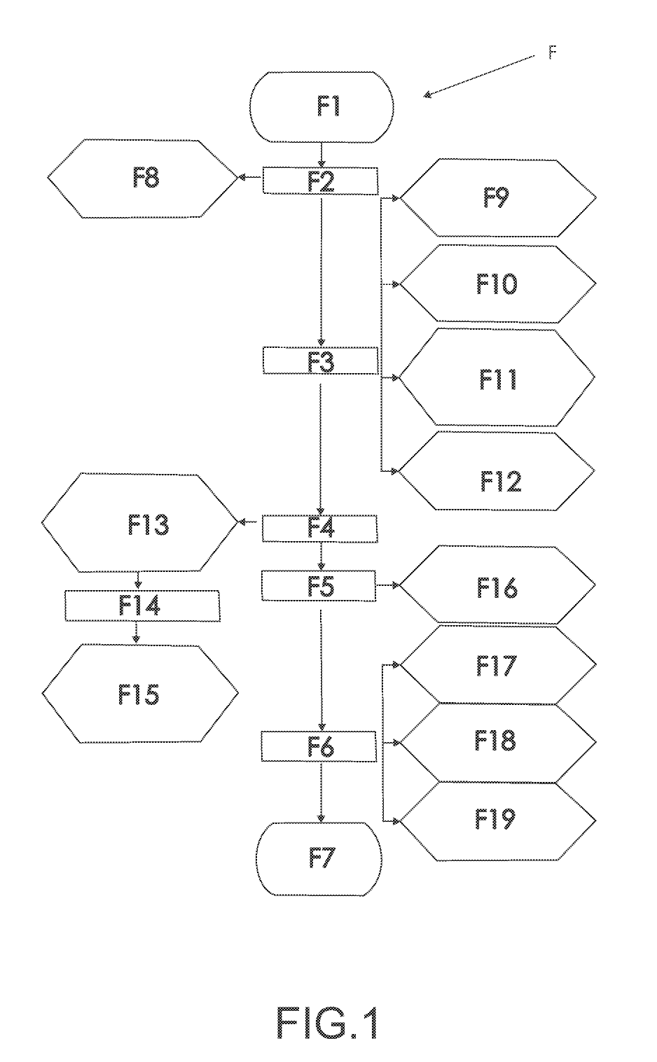

The process hereby proposed is depicted in FIG. 1, where the flowchart F presents the blocks relating to the various stages of the process with the following subtitles:

F1—Beginning—Raw Material Sugar Cane Molasses;

F2—Must Preparation;

F3—Sterilization;

F4—Inoculation;

F5—Propagation;

F6—Brightening;

F7—End—Obtainment the product: Purified Bacterial Cellulose.

In the same flowchart F depicted in FIG. 1, it still has the following blocks:

F8—Dilution with water to “Brix 10”;

F9—“Pumping of the must to sealed sterilization unit”;

F10—“Steam sterilization”;

F11—“Cooling of the must between 38 and 35° C. in the sterilization unit by exchange of natural heat with the environment or by active exchange from a cold water source”;

F12—“Pumping of the sterile must to the biodigestors”;

F13—“Collection of cell mass by mobile unit coupled by pipes to the biodigestors and transfer to the propagation biodigestor. Aliquot withdrawal for quality control”;

F14—“Quality Control”;

F15—“Quality Control. Collection of aliquots for inoculation in Erlenmeyer flasks, measurement of pH and microscopic evaluation of cell viability”.

F16—“Process of propagation in biodigestors with controlled temperature of the environment between 28° C. and 32° C.”;

F17—“Collection and transfer of polymer mass to the washing tank in flowing water, volume from 10 to 20 times the bacterial cellulose mass for a period from 6 to 24 hours until brown coloring is reached”;

F18—“Definitive brightening—successive washes in sodium hypochlorite solution at a concentration of 0.3% up to 3% in the ratio of 5 to 10 times the polymer mass volume until a pearlescent and translucent white coloring is obtained—period between 6 hours and 36 hours”;

F19—“After brightening, transfer to tanks with distilled water in the ratio of 5 to 10 times the polymer mass. Change every 6 hours to remove the hypochlorite residue, which is identified when the pH of the effluent reaches 6.2—period of 12 to 36 hours”.

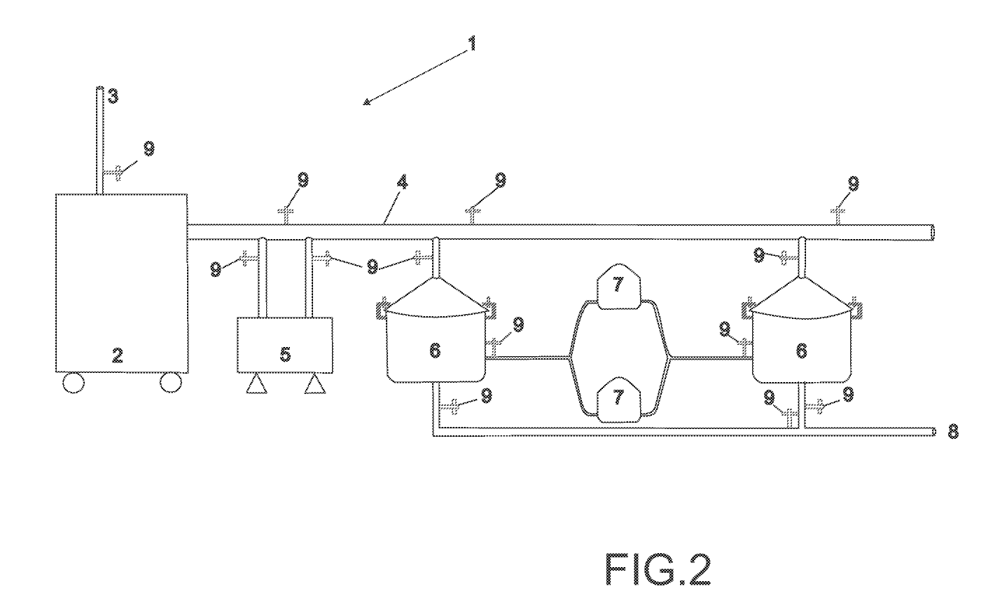

The FIG. 2 illustrates a diagram of the equipment used in this process and that configure the sterilization and propagation sealed unit, which is indicated, generally, by number reference 1, where can be seen the representation of sealed unit to production of hot water, steam and must sterilization 2.

The sealed unit for production of hot water, steam and must sterilization 2 is connected, by its upper region, to water and must receiving tube 3 and by its side, to washing, sterilization and feeding line 4.

In the same washing, sterilization and feeding line 4, it is arranged a pressurizing pump for water and must 5 and, in the sequence, it is the biodigestor 6.

The biodigestor 6 is connected to a mobile unit of cell mass transfer 7, to which is connected, on the other side, to a second biodigestor 6, wherein both biodigestors 6 are connected, by their respective upper portions, to the washing, sterilization and feeding line and, by their respective bottom portions, to a line 8 to exhaust tailings for collection of liquid wastes. The material flow over the unit 1, depicted in FIG. 2, is commanded by operating records 9.

The process of purification used for obtainment bacterial cellulose from crude polymer mass is safe, simple and fast, ensuring sustainability in all cycles of the process.

The process of scale production of bacterial cellulose in sterilization and propagation sealed unit and the process of purification of polymer mass obtained by said process allows the controlled obtainment of bacterial cellulose with high purity level for application in production of different products from purified cellulose for application in medicine, pharmacotechnical and cosmetic dermatology areas.

The cycle of processes using sugar cane derivatives such as sugar cane juice, treacle or molasses up to the purified bacterial cellulose is a technical-scientific innovation of economic impact.

The cellulose is obtained through polymerization of glucose via microbiologic from sugars of renewable sources, derivatives of sugar cane, such as molasses, concentrated syrup, sugar, sugar cane juice and the other sugars, such as fructose, derived from fruit and coconut water, and lactose from dairy products via microorganism Gluconoacetobacter hansenii LMSPE isolated from regional biodiversity and propagated on biodigestors (reactors).

In this process, the must, propagation source, is prepared from derivatives such as sugar cane molasses, concentrated sugar cane syrup, natural sugar cane juice obtained by crushing the sugar cane, preferably by easy of offering among other fruit derivatives such as coconut water or milk derivative serum obtained in the process of dairy products.

The concentration of must sugar is defined through the Brix scale and adjusted to a minimum value of 7.5 and maximum of 15.0, preferably in the optimal range of Brix 10, measured by a refractometer.

The adjustment of Brix to the determined range is obtained by must dilution with water or by concentration from a sugar source such as sucrose, glucose, fructose or lactose.

Preparation of must from sugar cane molasses flowchart, FIG. 1. A must aliquot with Brix adjusted to the determined scale is transferred to Erlenmeyer flasks and autoclaved and used to production and microbiological control.

For the scale production, the prepared must with Brix adjusted is transferred through pumping to a sealed sterilization unit with steam thermal source or electrical resistance.

After sterilization, the must is cooled still in the sterilization unit by heat natural exchange with the environment or by active exchange from a cold-water source.

The sterile must reaching the temperature between 38° C. and 35° is pumped into the biodigestors previously sterilized by water steam from sealed unit of steam and sterilization production.

To the biodigestors loaded with sterilized must and with temperature adjusted from 28° C. to 32° C. is added the propagation source cell mass, specific strain of the microorganism Gluconoacetobacter hansenii LMSPE, from a biodigestor whose fermentative process is already completed, the collection phase of bacterial cellulose.

For external control of biological characteristics and propagation potential of the cell mass, aliquots are inoculated in Erlenmeyrs with sterilized must.

At that time, cell mass aliquots are seeded into flasks with liquid culture medium and on plates with solid media to evaluate the stability and purity of the colonies.

In one of the aliquots, cell viability is evaluated by microscopic observation of the mobility of cells that should not be less than 25%.

The transfer of the cell mass among the biodigestors is performed by means of a sterile collector container connected to two biodigestors by two flexible tubes with taps previously sterilized by water steam flow from the sterilization unit.

One of the tubes connected to the collector container is coupled to the donor biodigestor with the complete fermentative process, and the other tube is connected to the receiver biodigestor with the newly sterilized must.

The container collector of sterile transfer is positioned uneven with the closed receiver biodigestor line by loading it with the biodigestor cell mass of the exhaust by aspirative flow.

After the collector load, the tap of the transfer tube is closed and the collector container is raised to a higher level than the receiver biodigestor, the propagation biodigestor, and the tap of the transfer tube is opened.

After the cell mass transfer, the tap of the transfer tube is closed and the system disconnected.

The biodigestors installed in the propagation room make heat external exchange direct with controlled temperature of the environment between 28° C. and 32° C. The stirring rotor is turned 24 hours after inoculation and adjusted to one evolution cycle per hour.

The biodigestor is discharged when an aliquot of must propagation reaches Brix less than 2 or pH less than 4. The process is continuous, transferring the cell mass of a donor or discharge biodigestor to another propagation, receiver biodigestor.

The polymer mass accumulated by natural flotation in the must surface is collected and transferred to the washing tank in a volume of flowing water from 10 to 20 times the bacterial cellulose mass for a period of 6 to 24 hours for withdrawal of must and sugars wastes until a polymer mass presents a light brown coloring.

The definitive brightening is obtained through successive washings until complete brightening in sodium hypochlorite solution in the concentration of 0.3% up to 3%, in the ratio of 5 to 10 times the volume of polymer mass, until a pearlescent and translucent white coloring is obtained, which occurs 6 hours and 36 hours.

After brightening, the bacterial cellulose mass is transferred to a tank of distilled water, also in the ratio 5 to 10 times the volume of polymer mass, which is changed each 6 hours, to withdraw all sodium hypochlorite residue used in the process of brightening identified when the pH of effluent reaches 6.2. The cycle occurs in a period from 12 to 36 hours.

The process results in scale obtainment, of a polymer mass, of pure and clarified bacterial cellulose with high content of nanocellulose. The process of scale production of bacterial cellulose with high content of micro- and nanofibrils in high productivity biodigestors is of innovative impact and economic interest.

1.-9. (canceled)

10. A process of scale production and purification of bacterial cellulose obtained by glucose polymerization from sugars of renewable sources via biotechnology through the propagation of Gluconoacetobacter hansenii LMSPE in reactors and obtainment of the purified cellulose for application in health, pharmacotechnical and cosmetic dermatology areas, wherein it uses sugars derived from sugar cane and it derivatives, including sugar cane juice, treacle, molasses, syrup and other sources also renewable such as dairy derivatives and coconut water through biotechnological way by Gluconoacetobacter hansenii LMSPE isolated from regional biodiversity, comprising the steps of: a) preparing the must;
b) sterilizing the must; c) inoculating the must; d) propagating; e) brightening) and f) obtaining the purified bacterial cellulose product.

11. The process of claim 10, wherein the must is adjusted, according to Brix scale, to a value between 7.5 and 15.0, considering as optimal the value of Brix 10, being this concentration value measured by a refractometer, wherein the Brix adjustment to certain range is obtained by diluting the must with water or by concentration from sugar source such as sucrose, glucose, fructose or lactose.

12. The process of claim 10, wherein must is fed to biodigestors participants of a sealed sterilization unit, being inoculated with the microorganism GLUCONOACETOBACTER HANSENII LMSPE thus leading to the propagation of this microorganism.

13. The process of claim 10, wherein the polymer mass accumulated by natural flotation in the must surface is collected and transferred to the washing tank in a volume of flowing water from 10 to 20 times to the bacterial cellulose mass for a period of 6 to 24 hours to withdraw the must and sugars wastes until the polymer mass presents a light brown coloring, being the definitive brightening obtained by successive washings until complete brightening in sodium hypochlorite solution at a concentration of 0.3% up to 3% in the ratio of 5 to 10 times the volume of polymer mass until a pearlescent and translucent white coloring is obtained, which occurs between 6 hours and 36 hours; after brightening, the bacterial cellulose mass is transferred to a tank of distilled water also in the ratio of 5 to 10 times the volume of polymer mass, which is exchanged each 6 hours to withdraw all sodium hypochlorite residue used in the process of brightening identified when the pH of effluent reaches 6.2.

14. The process of claim 10, wherein it employs a sealed sterilization and propagation unit, wherein a sealed unit is provided for production of hot water, steam and must sterilization; the sealed unit for production of hot water, steam and must sterilization is connected, by its upper region, to a water and must receiving tube and by its side to a washing, sterilization and feeding line; in the same washing, sterilization and feeding line it is arranged a pressurizing pump for water and must and in its sequence is the biodigestor; the biodigestor is connected to a mobile unit of cell mass transfer, which is connected, on the other hand, to a second biodigestor, wherein both biodigestors are connected, by their respective upper portions, to the washing, sterilization and feeding line and, by their respective bottom portions, to a line to exhaust tailings for collection of liquid wastes, being the material flow over the unit commanded by operating records.

15. The process of claim 14, further comprising the use of biodigestors of low depth and wide internal area to increase the conversion rate of polymer mass substrate, said biodigestors having flotation surface four times the height.

16. The process of claim 14, wherein it uses biodigestors (6) of low depth and wide internal area, where the same are distributed in series and coupled to a steam source forming a sealed sterilization and propagation unit (1).

17. The process of claim 14, wherein the biodigestors are distributed in series and coupled to a steam source forming a sealed sterilization and propagation unit, being further coupled to a sealed collector unit of cell mass transfer for propagation.

18. The process of claim 14, further comprising a sealed collector unit of cell mass transfer for propagation and obtainment of aliquots for quality control analysis of the cell mass without contamination.

19. A process comprising the steps of: (a) preparing a must from a sugar of a renewable source; (b) sterilizing the must; (c) inoculating the must; (d) propagating the must by use of Gluconoacetobacter hansenii LMSPE; (e) brightening; and (f) obtaining a purified bacterial cellulose product.
